(19) United States
(12) Patent Application Publication
(10) Pub. No.: US 2026/0174467 A1
(43) Pub. Date: Jun. 25, 2026

(54) Humeral Bone Preparation

(71) Applicant: Arthrex, Inc., Naples, FL (US)

(72) Inventors: Thomas DOONEY, JR., Naples, FL (US); Matthew T. PROVENCHER, Basalt, CO (US); Peter J. MILLETT, Naples, FL (US)

(73) Assignee: Arthrex, Inc., Naples, FL (US)

(21) Appl. No.: 19/426,196

(22) Filed: Dec. 19, 2025

Related U.S. Application Data
(60) Provisional application No. 63/737,859, filed on Dec. 23, 2024.

(51) Int. Cl. A61B 17/56; A61B 17/04; A61B 17/16

(52) U.S. Cl. CPC A61B 17/56; A61B 17/0401; A61B 17/16; A61B 2017/0404; A61B 2017/044; A61B 2017/0464; A61B 2017/564

(57) ABSTRACT

Surgical constructs methods of tissue fixation are disclosed. A textured structure on a bone surface provides increased friction and increased surface area for soft tissue coming down on it.

### CROSS-REFERENCE TO RELATED APPLICATIONS

This application claims priority to U.S. Provisional Patent Application No. 63/737,859 filed Dec. 23, 2024, the disclosure of which is hereby incorporated by reference in its entirety herein.

### BACKGROUND

The present disclosure relates to the field of surgery and, more particularly, to methods of surgical repairs.

### SUMMARY

Surgical constructs, assemblies, and kits are disclosed. A high friction bone preparation device provides a biologic surface to get down to bleeding bone for soft tissue to heal. A biologic bone surface may be prepared with a plurality of grooves or indentations provided in a direction about non-parallel to that of the soft tissue pull. A textured bone structure may be provided in various patterns to increase friction.

Methods of surgeries are also disclosed. In some implementations, a method of tissue fixation may be conducted by (i) preparing a textured structure on a bone surface using a cutting tool; and (ii) repairing soft tissue over the prepared bone surface. The repair compresses the soft tissue down into the textured structure to add additional strength and stability to the repair.

These and other features and advantages of this disclosure will become apparent and will be understood from the following detailed description of the various aspects of the disclosure taken in conjunction with the accompanying drawings.

### BRIEF DESCRIPTION OF THE DRAWINGS

FIG. 1 illustrates a humeral footprint undergoing bone preparation.

FIG. 2 illustrates another humeral footprint undergoing bone preparation.

FIG. 3 illustrates an anatomical view of a humeral footprint undergoing bone preparation.

FIG. 4 illustrates another anatomical view of a humeral footprint undergoing bone preparation.

FIG. 5(a)-(c) illustrate various views of a prepared humeral footprint.

FIG. 6 illustrates an exemplary instrument for textured bone surface.

FIG. 7 illustrates a textured bone surface formed with the instrument of FIG. 6.

FIG. 8 illustrates an exemplary instrument for another textured bone surface.

FIG. 9 illustrates a textured bone surface formed with the instrument of FIG. 8.

FIG. 10 illustrates an exemplary soft tissue repair.

### DETAILED DESCRIPTION

The present disclosure provides methods, surgical constructs, assemblies and systems for tissue repairs, for example, rotator cuff reconstruction.

A high friction bone preparation method and device provide a biologic surface to get down to bleeding bone for soft tissue to heal. A biologic bone surface may be a textured surface prepared with a plurality of grooves or indentations. The grooves may be provided in a direction about non-parallel to that of the soft tissue pull. The grooves may be provided in various patterns and/or geometries to increase friction.

During humeral footprint preparation, the surgeon's intent is to prepare the humeral surface to get down to bleeding bone to provide a biologic surface for soft tissue to heal down to. The bone preparation aids in soft tissue repair. The disclosure prepares the humeral footprint or bone to a “structured high friction surface” that increases the initial mechanical bond between the soft tissue and bone when repaired. The prepared surface may have grooves or similar structures cut so that they are perpendicular to the direction of soft tissue pull to aid in mechanical strength. The prepared surface may have grooves cut in a specific manner that forms unique, tortuous, non-uniform, non-flat patterns to increase friction. The soft tissue is repaired over the prepared surface. The repair compresses the soft tissue down into the grooves to add additional strength and stability to the repair.

The overall benefits of the disclosure are (i) increased initial repair strength due to the mechanical aspect of additional friction and (ii) more surface area for the soft tissue to heal to bone as the tissue may go down into the bone grooves to heal (all while still providing the same core benefits of traditional bone decortication).

The grooves may be prepared using a burr, rasp, punch, saw or any other bone cutting tool. The grooves may have various patterns and/or geometries. The grooves may be rounded, triangular, square, angled teeth, etc. or combinations thereof. The teeth may be cut at an angle to help grab the soft tissue. The tool trajectory may come “top-down” or from the side to prepare the grooves. The tool may prepare one groove at a time or a specific pattern of multiple grooves.

Methods of surgery are also disclosed. A method of altering a bone surface and increasing soft tissue to bone fixation may include inter alia the step of forming a textured structure onto an outer bone surface that has soft tissue come down on it, for increased friction and increased soft tissue fixation and retention. The textured structure may be a three-dimensional structure with increased surface area for soft tissue to adhere to bone. The method may further include forming a non-uniform, non-flat surface pattern onto the bone surface to increase adherence of the soft tissue to bone and to prevent the soft tissue from pulling away. The method may further include providing one or more grooves or indentations within the bone surface, wherein at least one groove may be cut in a direction about non-parallel to that of the soft tissue pull. The grooves may be cut in a direction about perpendicular to that of the soft tissue pull. The method may further include cutting the grooves to form a unique, non-uniform pattern to increase friction with soft tissue. The pattern may be a non-flat pattern. The pattern may be a tortuous pattern. The pattern may be a rough pattern.

A method of soft tissue repair may include inter alia the steps of (i) providing one or more textured structures within a bone surface; and (ii) providing soft tissue over the one or more textured structures of the bone. The textured structures may include one or more grooves and/or indentations, wherein at least one of the one or more grooves may be cut in a direction about non-parallel to that of the soft tissue pull. The method may further include compressing the soft tissue down into the one or more grooves. The method may further include securing the soft tissue to bone.

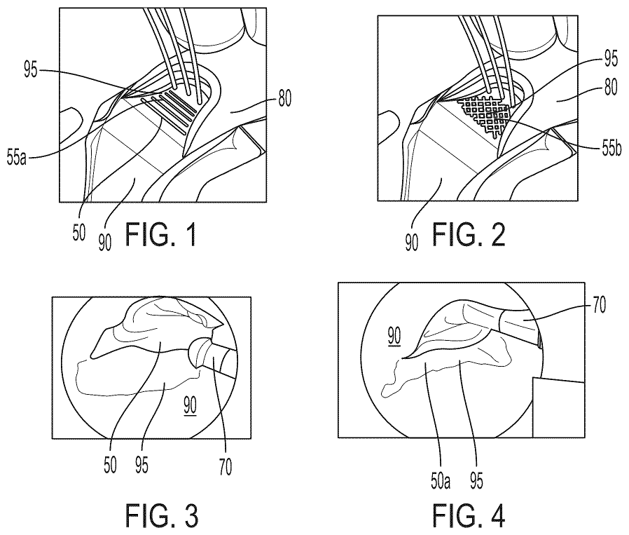

Referring now to the drawings, where like elements are designated by like reference numerals, FIGS. 1-10 illustrate various views of exemplary textured bone surface 95 and associated bone 90 and soft tissue 80 as part of repair 100.

FIGS. 1 and 2 illustrate exemplary textured bone surface 95 which may be a structure 95 formed of a plurality of grooves 50 provided within at least a surface of bone 90. For simplicity, the implementations detailed below will be illustrated with reference to a humeral bone surface for an exemplary rotator cuff repair 100. However, it must be understood that the disclosure is not limited to this exemplary-only implementation and also contemplates formation of textured structures and/or textured surfaces on any outer surface of a bone that requires (or that would benefit from) attachment of soft tissue to it.

Grooves 50 may be in the form of any indentations, dents, serrations and/or trenches, or similar structures formed within the bone surface to provide a pattern 55a, 55b as shown in FIGS. 1 and 2. The groove pattern may be preferably non-uniform to provide a non-flat, rough and coarse surface, to allow soft tissue 80 to securely adhere to the bone surface 95, to increase friction, and to prevent the soft tissue 80 from pulling away from the bone 90.

As shown in FIGS. 1 and 2, the grooves may be cut in a direction that is about non-parallel to the direction of soft tissue pull, to aid in mechanical strength. In some implementations, the prepared surface 95 may have grooves 50 cut in a direction about perpendicular to that of soft tissue 80. The grooves may be cut to form unique, tortuous, non-uniform, non-flat patterns to provide more friction and to increase the soft tissue retention within the pattern. As detailed below, the soft tissue 80 is repaired over the prepared textured surface 95. The repair compresses the soft tissue 80 down into the grooves 50 to add additional strength and stability to the repair.

FIGS. 3 and 4 illustrate anatomical views of a humeral footprint 90 undergoing bone preparation. Cutting instrument 70 forms textured surface 95 (textured three-dimensional structure 95) by cutting into bone 90 and forming grooves 50, 50a. Cutting instrument 70 may be a burr, rasp, punch, saw or any other bone cutting tool known in the art.

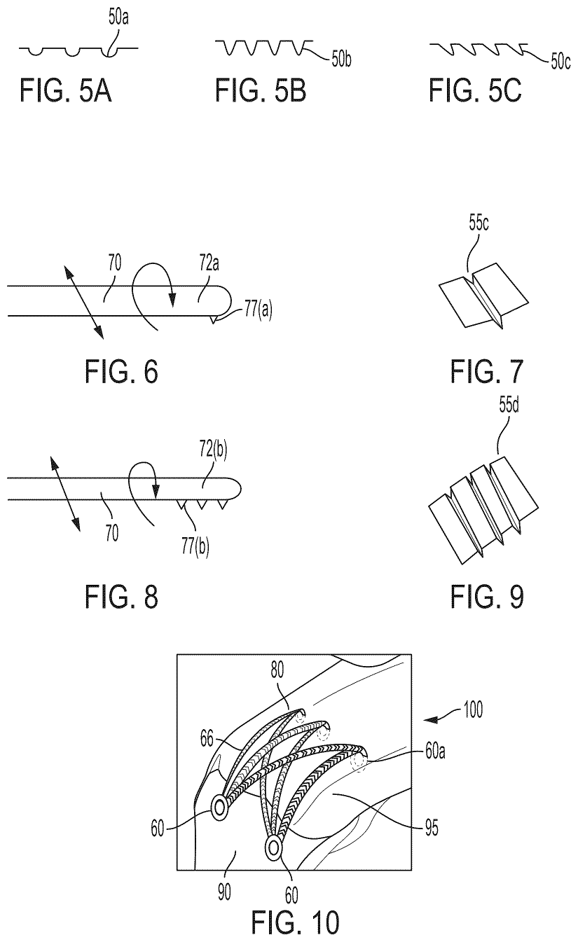

The grooves 50 may have various patterns and/or geometries. As depicted in FIG. 5(a)-(c), the grooves may be rounded 50a, triangular 50b, angled teeth 50c, square etc. or combinations thereof. The grooves may be cut to provide different patterns which in turn provide more friction and increased adherence of soft tissue 80 to bone 90.

Reference is now made to FIGS. 6-9 which illustrate various views of distal end 72 of cutting instrument 70 and corresponding groove formation. FIG. 6 illustrates distal end 72a of exemplary instrument 70 with a single cutting tooth 77a. FIG. 7 illustrates textured bone surface pattern 55c formed with the distal end 72a of cutting instrument 70 of FIG. 6.

FIG. 8 illustrates distal end 72b of exemplary instrument 70 having three exemplary cutting teeth 77b. FIG. 9 illustrates textured bone surface pattern 55d formed with the distal end 72b of cutting instrument 70 of FIG. 8. The teeth may be cut at an angle to help grab the soft tissue. The tool trajectory may come “top-down” or from the side to prepare the grooves. The tool 70 may prepare one groove at a time or a specific pattern of multiple grooves.

Cutting tool 70 may be provided with any number of cutting teeth or similar structures, to form any number of corresponding grooves. The cutting tool/instrument 70 may be employed from side to side (in a sagittal direction) or from top to down and may be provided with a depth stop. In some implementations, the cutting instrument 70 may be provided with a covering sheath or may be a self-guided instrument that has only cutting teeth coming out. In some implementations, the cutting instrument 70 may be in the form of a surgical saw with a depth stop, to allow the surgeon to easily control the groove formation and the dimensions of the as-formed pattern.

FIG. 10 illustrates soft tissue 80 secured over textured bone surface 95 of bone 90 with exemplary flexible strands 66 and fixation devices 60, 60a. Fixation devices 60, 60a may be knotted or knotless anchors (or combination thereof) so that repair 100 is a knotted or knotless repair (or combined repair).

The methods detailed above may be employed in various procedures for approximating or re-approximating soft tissue to bone, for example, shoulder rotator cuff repairs, capsulolabral reconstruction, SLAP repairs, as well as ankle, knee, elbow or foot repairs.

Cutting instrument 70 and associated fixation devices 60, 60a and flexible strands 66 described above may be included in a surgical kit, assembly, or system to simplify the surgeon's task of selecting a specific instrument and to aid in the overall surgical procedure. A surgical kit for an arthroscopic surgical repair may include one or more cutting instruments 70 (with various teeth configuration) as well as drills or additional bone-penetrating devices. The surgical kit may also include fixation devices 60, 60a (such as anchors, screws, suture-button constructs etc.) and flexible members 66 (such as sutures) to be employed in conjunction with the soft tissue repair.

A textured structure 95 on a bone 90 that has soft tissue 80 coming down on it is disclosed. The textured structure may be a three-dimensional structure with a non-uniform, non-flat surface pattern onto the bone surface to increase adherence of the soft tissue 80 to bone 90 and to prevent the soft tissue 80 from pulling away. The textured bone surface 95 may have one or more grooves 50, 50a, 50b, 50c. The grooves may form a pattern 55a, 55b, 55c, 55d on the surface 95. At least one groove may be cut in a direction about non-parallel to that of the soft tissue pull. Preferably, the grooves may be cut in a direction about perpendicular to that of the soft tissue pull to increase adherence and friction with soft tissue 80.

Textured bone structure 95 may be a non-flat, coarse three-dimensional surface area to increase the friction and soft tissue retention within the grooves or gaps of the structure. Textured bone structure 95 provides an increased surface area for the soft tissue 80 to adhere to bone 90. Textured bone structure 95 may have a non-uniform pattern.

Methods of altering a bone surface for increasing soft tissue to bone fixation may include inter alia the step of forming a textured structure 95 onto a bone surface 90 that has soft tissue 80 come down on it, for increased friction and increased soft tissue fixation and retention. The textured structure 95 may be a three-dimensional structure with increased surface area for soft tissue 80 to adhere to bone 90. The method may further include forming a non-uniform, non-flat surface pattern 55a, 55b, 55c, 55d onto the bone surface, to increase adherence of the soft tissue 80 to bone 90 and to prevent the soft tissue 80 from pulling away. The method may further include providing one or more grooves 50, 50a, 50b, 50c or similar structures within the outer bone surface, wherein at least one groove is cut in a direction about non-parallel to that of the soft tissue pull. The grooves may be cut in a direction about perpendicular to that of the soft tissue pull. The method may further include cutting the grooves 50, 50a, 50b, 50c to form a unique, non-uniform, non-flat, tortuous, rough pattern 55a, 55b, 55c, 55d to increase friction with soft tissue 80.

A method of soft tissue repair 100 may include inter alia the steps of (i) providing one or more textured surfaces 95 within an outer surface of a bone 90; and (ii) providing soft tissue 80 over the one or more textured surfaces 95 of the bone 90. The textured surfaces 95 may include one or more grooves 50, 50a, 50b, 50c or similar structures, wherein at least one of the one or more grooves may be cut in a direction about perpendicular to that of the soft tissue pull. The method may further include compressing the soft tissue 80 down into the one or more grooves 50, 50a, 50b, 50c so that the soft tissue follows the configuration of the grooves. The method may further include securing the soft tissue 80 to bone 90 with fixation devices 60, 60a and flexible strands 66.

In some implementations, a fixation device 60, 60a employed with repair 100 may be an all-suture soft anchor (soft suture anchor) provided with a soft anchor sleeve (sheath, tubular member) with two open ends and one or more flexible shuttling strands extending through the soft anchor sleeve (sheath). The at least two flexible strands may extend through the sleeve in similar or different directions and/or orientations and/or locations. The flexible strands may include high strength suture such as FiberWire® suture, TigerWire® suture, FiberTape® suture tape, among many others. The flexible sleeve with the one or more shuttling strands is secured into or onto bone 90, and the strands allow passing of additional flexible strands such as sutures and tapes to pass over soft tissue 80 and be secured into bone 90 and over the textured bone structure 95, to approximate soft tissue 80 to bone 90.

Fixation device 60, 60a may be any implant, button, anchor (for example, knotted anchor, knotless anchor, or all-suture anchor) or any device that confers secure attachment and fixation of soft tissue 80 to bone 90. The fixation device 60 may be a knotless anchor such as a two-piece Arthrex PushLock® anchor, disclosed in U.S. Pat. No. 7,329,272, or an Arthrex SwiveLock® anchor, disclosed in U.S. Pat. Nos. 8,012,174 and 9,005,246, the disclosures of both of which are fully incorporated by reference in their entirety herein. As noted, the fixation device 60 may be an all-suture soft anchor. Details of an exemplary soft suture anchor with a soft anchor sleeve (sheath or tubular member) and flexible shuttling strands are set forth, for example, in U.S. Pat. No. 10,849,734 issued Dec. 1, 2020, entitled “Methods of Tissue Repairs,” the disclosure of which is incorporated by reference in its entirety herein.

Flexible strands 66 may be formed of various flexible materials and strands such as round suture, flat suture, ribbon, or flat tape (for example, suture tape) or combination of suture and tape. Exemplary materials may include suture, silk, cotton, nylon, polypropylene, polyethylene, ultrahigh molecular weight polyethylene (UHMWPE), polyethylene terephthalate (PET), and polyesters and copolymers thereof, or combinations thereof. Flexible strands 66 may have cross-sections of various forms and geometries, including round, oval, rectangular, or flat, among others, or combination of such forms and geometries. In some implementations, flexible strands 66 may be formed of a high strength suture material such as FiberWire® suture, sold by Arthrex, Inc. of Naples, Fla., and described in U.S. Pat. No. 6,716,234, the disclosure of which is incorporated by reference herein. FiberWire® suture is formed of an advanced, high-strength fiber material, namely ultrahigh molecular weight polyethylene (UHMWPE), sold under the tradenames Spectra® (Honeywell International Inc., Colonial Heights, Va.) and Dyneema® (DSM N.V., Heerlen, the Netherlands), braided with at least one other fiber, natural or synthetic, to form lengths of suture material. Flexible strands 66 may be braided or multi-filament suture such as FiberTape® suture tape (as disclosed in U.S. Pat. No. 7,892,256, the disclosure of which is incorporated in its entirety herewith). Flexible strands 66 may include elastic material. Flexible strands 66 may consist essentially of elastic suture.

The term “high strength suture” is defined as any elongated flexible member, the choice of material and size being dependent upon the particular application. For the purposes of illustration and without limitation, the term “suture” as used herein may be a cable, filament, thread, wire, fabric, or any other flexible member suitable for tissue fixation in the body.

What is claimed is:

1. A textured bone structure on a bone surface for increased retention of soft tissue anatomically attached to the bone surface.

2. The textured bone structure of claim 1, wherein the bone surface is humeral surface and the soft tissue is rotator cuff.

3. The textured bone structure of claim 1, wherein the textured bone structure includes a plurality of grooves formed within the bone surface.

4. The textured bone structure of claim 3, wherein at least one of the plurality of grooves has a direction about non-parallel to a direction of soft tissue pull.

5. The textured bone structure of claim 3, wherein at least one of the plurality of grooves has a direction about perpendicular to a direction of soft tissue pull.

6. The textured bone structure of claim 3, wherein the plurality of grooves form a uniform or non-uniform pattern.

7. The textured bone structure of claim 6, wherein the uniform or non-uniform pattern increases retention of the soft tissue within the plurality of grooves.

8. The textured bone structure of claim 3, wherein the plurality of grooves have a round, circular, triangular or square cross-sectional view.

9. A method of soft tissue repair, comprising:
providing at least one textured structure on an outer surface of a bone; and
providing soft tissue over the at least one textured structure.

10. The method of claim 9, further comprising:
cutting one or more grooves into the bone surface to form the at least one textured structure; and
providing the soft tissue into the one or more grooves.

11. The method of claim 10, further comprising cutting the one or more grooves with a cutting instrument.

12. The method of claim 10, further comprising compressing the soft tissue into the one or more grooves so that the soft tissue contacts the one or more grooves.

13. The method of claim 12, wherein the soft tissue follows a contour of the one or more grooves.

14. The method of claim 10, further comprising attaching the soft tissue to the bone with at least one fixation device.

15. The method of claim 14, wherein the at least one fixation device is a screw, a button, a suture-button construct, or an anchor.

16. The method of claim 15, wherein the anchor is a soft suture anchor comprising a flexible tubular sleeve or sheath and a plurality of flexible strands extending through a passage of the flexible tubular sleeve or sheath.

17. The method of claim 14, wherein the at least one fixation device is an all-suture soft anchor.

18. The method of claim 17, wherein the at least one fixation device is a hard body anchor.

19. The method of claim 9, wherein the soft tissue repair is rotator cuff repair.

20. A method of surgical repair, comprising increasing on an outer surface area of a bone surface undergoing a soft tissue repair.

21. The method of claim 20, further comprising:
forming a three-dimensional pattern within the bone surface to increase the outer surface area; and
compressing soft tissue into the outer surface area.

22. The method of claim 21, wherein the three-dimensional pattern is formed by cutting a plurality of grooves or trenches within the bone surface.

23. The method of claim 21, further comprising securing the soft tissue over the three-dimensional pattern with one or more fixation devices.
